(19) United States
(12) Patent Application Publication
(10) Pub. No.: US 2018/0264024 A1
(43) Pub. Date: Sep. 20, 2018

(54) SUBLINGUAL ADMINISTRATION OF VITAMIN B12 DISPERSED IN A HYDROPHOBIC CONTINUOUS PHASE

(71) Applicant: Klaas Alouis Riepma, Valkenburg (NL)

(72) Inventor: Klaas Alouis Riepma, Valkenburg (NL)

(21) Appl. No.: 15/761,146

(22) PCT Filed: Sep. 23, 2016
(86) PCT No.: PCT/NL2016/050659

§ 371 Date: Mar. 19, 2018

(30) Foreign Application Priority Data
Sep. 25, 2015 (NL) 2015506

(51) Int. Cl. A61K 31/714; A61K 9/00; A61K 9/10; A61K 47/44; A61P 25/28; A61P 25/06; A61P 37/00

(52) U.S. Cl. CPC A61K 31/714; A61K 9/006; A61K 9/10; A61P 37/00; A61P 25/28; A61P 25/06; A61K 47/44

(57) ABSTRACT

Vitamin B12 for use in the treatment of a condition in a subject is administered sublingually. The vitamin B12 is dispersed in a hydrophobic continuous phase, such as oil or fat. The condition to be treated is vitamin B12 deficiency.

### CROSS-REFERENCE TO RELATED APPLICATIONS

This application is the National Stage of International Application No. PCT/NL2016/050659, filed Sep. 23, 2016, which claims the benefit of Netherlands Application No. NL 2015506, filed Sep. 25, the contents of which is incorporated by reference herein.

### FIELD OF THE INVENTION

The present disclosure relates to the field of oral administration forms containing vitamin B12 for use in treating a condition in a subject, wherein the condition preferably is vitamin B12 deficiency.

### BACKGROUND OF THE INVENTION

Vitamin B12 is a vitamin that plays a role in mammalian growth, hematopoiesis, production of epithelial cells and maintainance of the nervous system. It is quite water-soluble and thus could be expected to be easily available to human subjects. However, the absorption from the gut of normal dietary amounts of vitamin B12 is believed to be dependent on gastric Intrinsic Factor (GIF), and the loss of Intrinsic Factor leads to vitamin B12 deficiency. The loss of ability to absorb vitamin B12 (B12) is the most common cause of adult B12 deficiency. Such a loss may, for example, be due to pernicious anemia (with loss of Intrinsic Factor) or to a number of other conditions that decrease production of gastric acid, which also plays a part in absorption of B12 from foods. Deficiency is most significantly linked to inadequate absorption rather than low consumption, as those who consume high amounts of vitamin B12 may still experience deficiency as evidenced by a low blood concentration.

Vitamin B12 deficiency results in various undesirable conditions such as fatigue, depression, poor memory, etc. Other causes of vitamin B12 deficiency include atrophic gastritis (a thinning of the stomach lining), surgery in which part of the stomach and/or small intestine is removed, conditions affecting the small intestine (such as Crohn's disease, celiac disease, bacterial growth, or a parasite), excessive alcohol consumption, autoimmune disorders (such as Graves' disease or systemic lupus erythematosus) and drug abuse.

Pharmaceutical compositions containing vitamin B12 are known in the art, for example from U.S. Pat. No. 5,801,161 to Merkus, which discloses an intranasal spray. Such a pharmaceutical composition is brightly red, with as the concomitant disadvantage that any fluid of the pharmaceutical composition running from the nose will give the appearance of a bloody nose.

Sharabi et al. (2003 J. Clin Pharmacol., 56, 635-638) disclose tablet formulations that are administered orally or sublingually for correcting vitamin B12 deficiency. However, poor bioavailability of vitamin B12 is demonstrated.

WO2014084736 discloses a pharmaceutical composition comprising vitamin B12 dispersed in a hydrophobic continuous phase which is administered intranasally. The administration form of WO2014084736 also leaves room for improvement.

The object of the present disclosure is to provide an administration form with good bioavailability of vitamin B12, and which is easy and convenient in use.

### SUMMARY OF THE INVENTION

The present disclosure provides an oral administration form characterized in that vitamin B12 is dispersed in a hydrophobic continuous phase. Surprisingly it has been found that oral administration of such a dispersion of solid vitamin B12 (crystalline or amorphic particles) in a hydrophobic continuous phase displays very satisfactory bioavailability.

### DETAILED DESCRIPTION OF THE INVENTION

The vitamin B12 can be comprised in a pharmaceutical composition which e.g. may be in the form of a drink, mouth drops, or sublingual drops. In the present application, the term vitamin B12 includes cyano-cobalamin, hydroxo-cobalamin, methyl-cobalamin, 5′-deoxyadenosyl-cobalamin, aquacobalamin, glutathionyl-cobalamin and nitrilocobalamin, including the pharmaceutically acceptable salts thereof, and including mixtures thereof. In general, the concentration of vitamin B12 in the hydrophobic continuous phase is between 0.01-50/75 wt. % with respect to the weight of the hydrophobic continuous phase.

According to a favourable embodiment, the hydrophobic continuous phase is chosen from at least one of i) fat, ii) fatty acids, and iii) wax. Thus, a hydrophobic environment for vitamin B12 is provided. In general, the fatty acids have a length of the carbon chain of at least 6.

According to a favourable embodiment, the hydrophobic continuous phase is (edible) oil/fat. The term “hydrophobic” is clear to the skilled person and means that mixing a liquid continuous phase with water forms an emulsion (with or without emulsifier). A hydrophobic phase typically has a water solubility of below 500, 250, 100, 50, 10, preferably below 5, or 1 mg/L pure water at room temperature, i.e. 20 degrees Celcius. If the hydrophobic continuous phase comprises more than one constituent, the average of their water solubility is considered, taking into account their relative wt. % with respect to the hydrophobic continuous phase as a whole.

Such a liquid or fatty pharmaceutical composition is convenient to administer and results in high absorption of vitamin B12 based on concentration in the blood of a human subject.

According to a favourable embodiment, the hydrophobic continuous phase is anhydrous. This promotes the release of vitamin B12 from the pharmaceutically acceptable carrier. Anhydrous, within the context of the present disclosure, means a water content of less than 5 wt. %, preferably less than 1 wt. % and more preferably with less than 0.2 wt. % with respect to the weight of the hydrophobic continuous phase.

According to a favourable embodiment, the hydrophobic continuous phase comprises methylcobalamin or a pharmaceutically acceptable salt thereof as vitamin B12.

Methylcobalamin is considered a powerful drug but because it decomposes easily this value has not been realized in oral pharmaceutical compositions according to the prior art as it cannot be stored or pharmaceutical compositions have to be kept frozen. The hydrophobic pharmaceutical composition according to the present disclosure will benefit from improved stability, in particular for methylcobalamin. Without wishing to be bound to any particular theory, it is believed that the fact that vitamin B12 is present as particles reduces its sensitivity to degradation. It is preferred that at least 25 wt. % of vitamin B12 is methylcobalamine or a pharmaceutically acceptable salt thereof, with respect to the weight of the hydrophobic continuous phase.

According to a favourable embodiment, the concentration of vitamin B12 is in the range of 0.05-10 wt. % preferably between 0.4-8 wt. %, with respect to the hydrophobic continuous phase.

Generally, the amount administered orally will be 25-1500 μl, preferably 50-1000 μl, or 50-500 μl (or 50-2500, or 75-5000 μl).

According to a favourable embodiment, vitamin B12 is colloidally dispersed. Such a pharmaceutical composition is stable for longer periods.

As is clear, the present disclosure relates to vitamin B12 dispersed in a hydrophobic continuous phase, for use in the treatment of a condition in a subject, wherein vitamin B12 is administered orally (or sublingually), and wherein the condition for example is vitamin B12 deficiency. Subligual administration can be seen as the pharmacological route of administration by which the vitamin B12 diffuses into the blood through tissues under the tongue. Accordingly, the present disclosure relates to a method of treating a subject in need thereof comprising administering, orally or sublingually, the vitamin B12 dispersed in a hydrophobic continuous phase.

However, where in the present disclosure reference is made to oral administration, rectal administration is a viable alternative. The preferred embodiments discussed above are equally applicable to this use, are included by reference for this use, and are not repeated for the sake of brevity only.

It has been found that such a dispersion of solid vitamin B12 particles (crystalline or amorphic) in a hydrophobic continuous phase displays very satisfactory bioavailability. The vitamin B12 deficiency is any condition where an increased level would be of benefit to the subject, which can be a human or an animal. It may be a condition chosen from autism spectrum disorder, fatigue, memory deficiency, ALS, Alzheimer, deficiency caused by drug abuse, thinning of the stomach lining, vitamin B12 deficiency after surgery in which part of the stomach and/or small intestine is removed, Crohn's disease, celiac disease, Graves' disease, systemic lupus erythematosus and migraine.

Finally, the present disclosure relates to a method of treating a human subject suffering from a condition chosen from vitamin B12 deficiency, autism spectrum disorder, fatigue, memory deficiency, ALS, Alzheimer, deficiency caused by drug abuse, thinning of the stomach lining, vitamin B12 deficiency after surgery in which part of the stomach and/or small intestine is removed, Crohn's disease, celiac disease, Graves' disease, systemic lupus erythematosus and migraine, wherein a pharmaceutical composition according to the present disclosure is administered orally.

The method avoids what would appear like a bloody mouth due to the intense red colour of vitamin B12. The disclosure will now be illustrated with reference to the example section below

### EXAMPLE 1

A composition of 200 mg methyl cobalamin in 10 g coconut fat, and further containing 1 wt. % pepper mint oil was prepared. The composition was administered to a volunteer via the oral route. The volunteer took 1.2 g of the composition (thus containing 20-25 mg Vitamin B12) and allowed it to melt under the tongue for 1 minute, after which the composition was swallowed.

Blood values were measured before and 1 hour after administration.
Vitamin B12 content before:
643 pmol/l
Vitamin B12 content after:
>1476 pmol/l

The above values demonstrate that Vitamin B12 is absorbed very well when administered orally in a hydrophobic continuous phase. The degree of absorption is surprisingly better when compared to oral administration via tablet formulation. For example, Sharabi et al. (2003 J. Clin Pharmacol., 56, 635-638) demonstrate an increase in Vitamin B12 blood levels of only 10 pmol/l per day for tablet formulations.

### EXAMPLE 2

Three different products were tested for best vitamin B12 absorption by a volunteer:
(1) a commercial tablet with Hydroxocobalamin (chloride), administered orally;
(2) a liposomal product containing Hydroxococobalamin (chloride) in 10 ml solution build into liposomes (containing water), administered sublingually; and
(3) hydroxocobalamin (chloride) in sesame oil containing the vitamin B12 mixed into sesame oil, administered sublingually.

The sublingual products were held in the mouth for 5 minutes. After these minutes the products were spit out and the mouth rinsed with water. At t=0 minutes and after 1 hr blood samples were taken and the amount of vitamin B12 measured in the blood.

| | Administration | | |
|---|---|---|---|
| Product | route | T = 0 | T = 60 minutes |
| Hydroxycobalamine 10 mg | oral | 255 pmol | 268 pmol |
| in a tablet | | | |
| Hydroxocobalamin 10 mg | sublingual | 317 | 365 |
| in a liposomal | | | |
| solution | | | |
| Hydroxocobalamin 10 mg | sublingual | 412 | 725 |
| in sesame oil | | | |

It can be seen that all products do result in an absorption of Vitamin B12. The best absorption is found with a hydrophobic basis (sesame oil). Further, it shows that the best absorption is achieved when vitamin B12 is administered sublingually in a hydrophobic suspension.

1. Vitamin B12 for use in the treatment of a condition in a subject, wherein the vitamin B12 is administered sublingually, and wherein the vitamin B12 is dispersed in a hydrophobic continuous phase.

2. Vitamin B12 according to claim 1, wherein the hydrophobic continuous phase is chosen from at least one of i) fat, ii) fatty acids, and iii) wax.

3. Vitamin B12 according to claim 1, wherein the hydrophobic continuous phase is oil.

4. Vitamin B12 according to claim 1, wherein the hydrophobic continuous phase is anhydrous.

5. Vitamin B12 according to claim 1, wherein the vitamin B12 is chosen from at least one of cyano-cobalamin, hydroxo-cobalamin, methyl-cobalamin, 5′-deoxyadenosyl-cobalamin, aquacobalamin, glutathionyl-cobalamin and nitrilocobalamin.

6. Vitamin B12 according to claim 1, wherein the concentration of vitamin B12 in the hydrophobic continuous phase is in the range of 0.05 wt. % to 10 wt. %.

7. Vitamin B12 according to claim 1, wherein vitamin B12 is colloidally dispersed in the hydrophobic continuous phase.

8. Vitamin B12 according to claim 1, wherein the subject is a human subject or an animal subject.

9. Vitamin B12 according to claim 1, wherein the condition is chosen from vitamin B12 deficiency, autism spectrum disorder, fatigue, memory deficiency, ALS, Alzheimer, deficiency caused by drug abuse, thinning of the stomach lining, vitamin B12 deficiency after surgery in which part of the stomach and/or small intestine is removed, Crohn's disease, celiac disease, Graves' disease, systemic lupus erythematosus and migraine.

10. Vitamin B12 according to claim 1, wherein the concentration of vitamin B12 in the hydrophobic continuous phase is between 0.4 wt. % and 8 wt. %.
